(19) United States
(12) Patent Application Publication
(10) Pub. No.: US 2010/0099085 A1
(43) Pub. Date: Apr. 22, 2010

(54) Serum DNA Methylation Screening for Cancer

(76) Inventors: Richard Babaian, Houston, TX (US); Herbert Fritsche, Houston, TX (US); Li-Ying Yang, Bellaire, TX (US)

Correspondence Address: WILLIAMS, MORGAN & AMERSON, 10333 RICHMOND, SUITE 1100, HOUSTON, TX, 77042, US

(21) Appl. No.: 12/376,148

(22) PCT Filed: Jul. 30, 2007
(86) PCT No.: PCT/US07/16970

§ 371 Date: Nov. 19, 2009

Related U.S. Application Data
(60) Provisional application No. 60/821,292, filed on Aug. 3, 2006.

(51) Int. Cl. C12Q 1/68

(52) U.S. Cl. 435/6

(57) ABSTRACT

We disclose a method of screening a mammal for a cancer associated with methylation of promoters of tumor-suppressor genes by collecting a sample of a body fluid or tissue of the mammal containing free DNA; isolating the free DNA from the sample; amplifying at least a portion of the promoter of each of a plurality of turner-suppressor genes associated with the cancer from the free DNA, to yield a plurality of amplified promoters; and quantifying methylation of each of the plurality of amplified promoters, to yield a methylation quantity. The method can have a high sensitivity and high specificity and can thus reduce the proportion of unneeded biopsies. It can also be performed with minimal invasiveness and discomfort to a patient.

### BACKGROUND OF THE INVENTION

The present disclosure relates generally to the field of cancer screening. More particularly, it concerns methods of cancer screening that possess both high sensitivity and high specificity.

In the United States and other developed countries, cancer strikes about 0.5% of the population in any given year and causes about 25% of all deaths. Prostate cancer is the most commonly diagnosed malignancy in American men and the third most common cause of male cancer deaths in the United States. The early detection of this disease is currently based upon the use of the serum prostate specific antigen (PSA) test (1, 2). However, the PSA test has its limitations. The test yields high incidences of false negative and false positive rates that ranges from 20% to 30% and 70% to 80%, respectively, and low specificity of 25-30% at a fixed sensitivity of 90% (3, 4). As a result, about 75% of men who undergo a prostate biopsy after being observed to have high PSA levels do not have prostate cancer and have undergone the prostate biopsy needlessly. Such limitations have lead to proposals of several enhanced measurements for the PSA test, including the use of percent free-PSA, complexed PSA, PSA density (total PSA/prostate gland volume) and PSA velocity (change in PSA over) time) (5-10). Unfortunately, all of these attempts fail to demonstrate a clinically significant benefit. The controversy over the PSA test has grown to the point that, in 2004, Stamey declared the “PSA Era” to be over (11). It is desirable to develop novel markers to improve the early detection of cancer, such as prostate cancer.

DNA methylation involves the addition of a methyl group to the 5-carbon of cytosine in cytosine-guanidine phosphodiester-linked (CpG) dinucleotides. DNA methylation is essential not only for normal mammalian development but also for the maintenance of cellular homeostasis. CpG dinucleotides are rarer in vertebrate genomes (about 1% of all dinucleotides) than would be expected by chance (about 6.25% of all dinucleotides, if the cytosine and guanidine content of the genome totals 50%). However, about half of all human gene promoter regions contain CpG islands, which are regions of at least 300 base pairs that have a CpG content greater than that expected by chance. Hypermethylation of CpG islands in the promoter regions of genes is associated with transcriptional silencing and aberrant DNA methylation is a frequent event in human cancers (12).

Aberrant DNA methylation as a molecular marker has recently been investigated in prostate cancer. The first report of CpG hypermethylation in prostate cancer demonstrated a significant incidence of hypermyethylation at the GSTP1 promoter region (13). This observation found at the tissue level has been confirmed by other investigators with a reported incidence of approximately 85% (13-16). The work performed in the cited references involved extraction of DNA from prostate cancer cells. However, studies on promoter hypermethylation in body fluids are limited. Also, there has not yet been a consensus regarding the incidence of hypermyethylation at the GSTP1 promoter region in non-malignant prostate tissue.

### SUMMARY OF THE INVENTION

In one embodiment, the present disclosure relates to a method of screening a mammal for a cancer associated with methylation of promoters of tumor-suppressor genes by collecting a sample of a body fluid or tissue of the mammal containing free DNA; isolating the free DNA from the sample; amplifying at least a portion of the promoter of each of a plurality of tumor-suppressor genes associated with the cancer from the free DNA, to yield a plurality of amplified promoters; and quantifying methylation of each of the plurality of amplified promoters, to yield a methylation quantity.

The method can have a high sensitivity and high specificity and can thus reduce the proportion of unneeded biopsies. It can also be performed with minimal invasiveness and discomfort to a patient.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. A representative methylation-specific PCR (MS-PCR) analysis of GSTP1, RASSF1A, RARβ, APC, and CDH1 with serum DNA from patients with prostate tumors. PCR products were separated on 2.0% agarose gels and were then visualized with the ethidium bromide staining. PC, designated as the positive control for the methylated form of the gene, was the normal lymphocyte DNA treated with Sss1 methyltransferase before the bisulfate modification. NL, the negative control for the unmethylated form, was the normal lymphocyte DNA. H₂O represents the DNA-free control. Lanes 4-7 were serum DNA from representative patients with low-grade prostate tumors; lanes 8-11 were serum DNA from representative patients with high-grade prostate tumors. MWK, molecular weight marker of o 100-bp or 25-bp DNA ladder. The presence of the bands in the lanes marked with M indicates the presence of methylated genes. In contrast, the presence of the bands in the lanes marked with U indicates the presence of unmethylated genes. The gene is considered as hypermethylated as long as a visible M band is detected, either present alone or co-present with U bands.

FIG. 2. Methylation analysis of GSTP1, RASSF1A, RARβ, APC, and CDH1 genes with serum DNA from men with and without prostate tumor. Free DNA was isolated from sera of men with stage A/B disease (A) or with the stage D disease (B) and from sera of men with negative biopsies (C). The isolated DNA was subjected to the MS-PCR analysis. Filled box represents the sample in which the specific gene is methylated; the open box with “-” o represents the sample containing unmethlylated gene.

FIG. 3. Methylation frequencies of GSTP1, RASSF1A, RARβ, APC, and CDH1 genes in men with and without prostate cancer. The methylation frequency is defined as the percent of the gene methylated in the total number of subjects tested. The percent of the methylation frequency for each gene was analyzed in 52 cancer patients (black box) and 35 non-cancer individuals (open box). n=sample number examined; P, the significance of difference between cancer and non-cancer groups.

FIG. 4. Methylation status of GSTP1, RASSF1A, and CDH1 genes in men with confined prostate cancer and men with a negative biopsy. The methylation status of the 3 selected genes was analyzed by MS-PCR using free DNA isolated from sera of 21 men (A) o who had confined prostate cancer and 32 men who were biopsy negative (B). Filled box represents the sample in which the specific gene is methylated; open box with “-” represents the sample containing an unmethlylated gene.

FIG. 5. Methylation frequencies of GSTP1, RASSF1A, and CDH1 genes in men with and without prostate cancer. The percent of methylation frequency for each gene was analyzed in 73 cancer patients (black box) and 67 non-cancer individuals (open box). n=sample number examined; P, the significance of difference between cancer and non-cancer groups.

FIG. 6. Comparison of the methylation index between the cancer and non-cancer groups. The methylation index (MI) is defined as the fraction of genes methylated of the 3 genes tested. The MI was analyzed in a total of 140 men with all PSA ranges (A) and in 119 men whose preoperative serum PSA level was <10 ng/ml (B). black box, cancer; open box, non-cancer; n=number of samples analyzed; P, the significance of difference between the means of the cancer and non-cancer group.

FIG. 7. Block diagram of a system screening a mammal for a cancer associated with methylation of promoters of tumor-suppressor genes.

### DESCRIPTION OF ILLUSTRATIVE EMBODIMENTS

In one embodiment, the present disclosure relates to a method of screening a mammal for a cancer associated with methylation of promoters of tumor-suppressor genes by collecting a sample of a body fluid or tissue of the mammal containing free DNA; isolating the free DNA from the sample; amplifying at least a portion of the promoter of each of a plurality of tumor-suppressor genes associated with the cancer from the free DNA, to yield a plurality of amplified promoters; and quantifying methylation of each of the plurality of amplified promoters, to yield a methylation quantity.

Any cancer associated with methylation of promoters of tumor-suppressor genes can be screened for by the method. In one embodiment, the cancer is prostate cancer. When the cancer is prostate cancer, the plurality of tumor-suppressor genes can be selected from the group consisting of GSTP1, RASSF1A, RARβ, APC and CDH1. In one embodiment, the plurality of tumor-suppressor genes associated with the cancer are selected from the group consisting of GSTP1, RASSF1A, and CDH1.

Any mammal can be the subject of the method. In one embodiment, the mammal is Homo sapiens. Other mammals that have economic (e.g., cow, sheep, pig) or esthetic (e.g., cat, dog, horse) utility can be the subject of the method, as well.

Any body fluid or tissue can provide the sample on which the amplifying step is performed. In one embodiment, the body fluid or tissue is selected from the group consisting of blood and urine. Samples of blood or urine are readily collectable with minimal discomfort and inconvenience to the mammal. The presence of free DNA in blood has been known since the 1940s. It has been hypothesized that free DNA enters blood through intravascular cell death or from circulating phagocytes that have ingested a cell.

Collecting the sample can be performed by techniques known to the skilled artisan having the benefit of the present disclosure. Such techniques will vary depending on the body fluid or tissue to be sampled, among other considerations.

In the isolating step, the free DNA in the sample can be isolated from other components of the sample by any appropriate technique, such as those well-known in the art.

In the amplifying step, the portion of the promoter of each of a plurality of tumor-suppressor genes can be a portion that contains at least one CpG island. Amplifying can be performed by techniques known to the skilled artisan having the benefit of the present disclosure. The amplifying step yields amplified promoters or amplified portions or promoters, which herein are all encompassed by the term “amplified promoters.”

In one embodiment, amplifying comprises methylation-specific polymerase chain reaction (MS-PCR). In another embodiment, amplifying comprises MS-PCR and pyrosequencing.

As is known in the art, bisulfite treatment of DNA converts unmethylated cytosine (C) to uracil (U) and leaves methylated cytosine (^{m}C) unchanged. In MS-PCR of bisulfite-treated DNA, the well-known polymerase chain reaction is modified by the use of two sets of primers, one containing CpG dinucleotide(s) which will only hybridize with the methylated form of the region to be amplified, and a second free of CpG dinucleotide(s). In its most basic form, MS-PCR can only provide a binary quantification (absence or presence) of methylation in the amplified region. More advanced forms of MS-PCR can report the relative number of methylated to unmethylated amplicons in the sample.

In one embodiment, when MS-PCR is used in amplifying, quantifying comprises dividing the number of methylated amplified promoters by the total number of promoters subjected to amplification. This quotient may be termed the “methylation index” or “MI.” A “methylated amplified promoter” is a promoter which is amplified by methylation-specific primers, regardless of whether an unmethylated form of the same promoter is amplified.

PCR of bisulfite-modified DNA amplifies methylated cytosine (^{m}C) to (C) and uracil resulting from conversion of unmethylated cytosine will be amplified to thymine (T). Thus, the methylated and unmethylated cytosine can be discriminated as a C/T SNP, which can be analyzed by DNA sequencing and quantified. Pyrosequencing is known in the art; in summary, it employs enzymatic cascade reactions to produce light for every nucleotide incorporated into a nascent sequence and yields a pyrogram with quantitative measurement of the incorporation event (peak heights of light intensity). Pyrosequencing using a bisulfite-modified, PCR-amplified ssDNA template, allows calculation of the degree of methylation based on the C:T peak heights. Pyrosequencing allows not only accurate quantification of the degree of methylation but also analysis of the site-specific CpG methylation pattern.

The sequence context and the light intensity of the pyrogram allow identification of the methylation status of each CpG site and quantification of the degree of methylation calculated from the peak heights of cytosine (C) and tyrosine (T):

% methylation=[C peak height/(C peak height+T peak height)]×100

In a further embodiment, the method can further comprise comparing the methylation quantity to a threshold quantity. For example, when quantifying yields a methylation index, a particular value of the methylation index can indicate a high probability that the mammal suffers from the cancer which is being screened. For example, in screening for prostate cancer, when the GSTP1, RASSF1A, and CDH1 promoters are examined, a methylation index of at least ⅔ indicates a high probability (about 85%) that the patient suffers from prostate cancer.

For another example, when quantifying yields a % methylation, a particular value of % methylation can indicate a high probability that the mammal suffers from the cancer which is being screened.

In another embodiment, the present disclosure relates to a system for screening a mammal for a cancer associated with methylation of promoters of tumor-suppressor genes, comprising:

means for collecting a sample of a body fluid or tissue of the mammal containing free DNA;

means for isolating the free DNA from the sample;

means for amplifying at least a portion of the promoter of each of a plurality of tumor-suppressor genes associated with the cancer from the free DNA; and

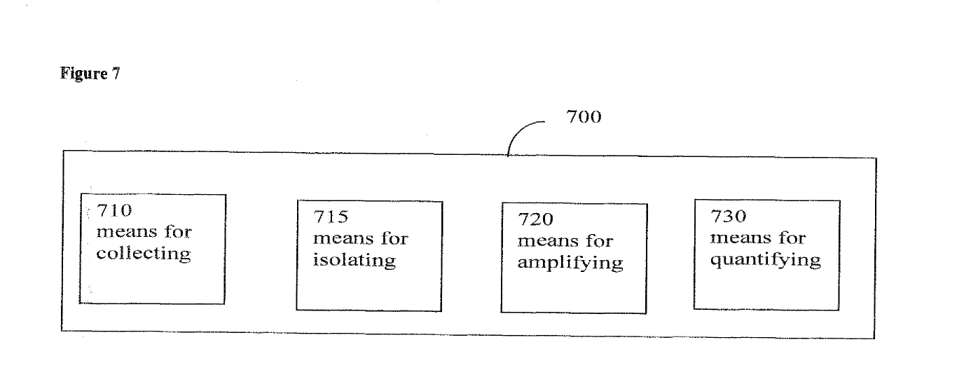

means for quantifying methylation of each of the plurality of amplified promoters. An example of such a system 700 is shown in FIG. 7.

The collecting means 710 can vary depending on the body fluid or tissue to be sampled. A syringe and hypodermic needle can be used to collect blood. A specimen cup can be used to collect urine.

The isolating means 715 means can comprise a kit for isolating free DNA from a sample.

The amplifying means 720 can comprise a PCR machine. An exemplary PCR machine is the PTC-200 DAN Engine (MJ Research, Watertown, Mass.). The amplifying means can also comprise a device or kit for extraction of DNA from the collected sample and a reaction vessel for bisulfite treatment of extracted DNA.

The quantifying means 730 can comprise devices for rendering amplified DNA visible to the human eye, such as a gel electrophoresis apparatus, along with the use of a DNA-specific dye, such as ethidium bromide, and a lamp capable of rendering the dyed DNA visible. Another quantifying means can be a pyrosequencing device, as are known in the art.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example

### Summary

Using the methylation index (MI) as the measurement of the extent of methylation among the genes, we evaluated the sensitivity and specificity of the MI as a diagnosis biomarker. In addition, the relationship between MI and PSA level, and between the MI and clinical characteristics of cancer patients such as the disease stage and Gleason sum were also assessed.

Materials and Methods

Serum samples and materials: Serum samples collected from men having signed an informed consent to have their blood stored in a prostate serum bank were used. A total of 140 retrospective serum samples including 77 men with prostate cancer and 67 men with negative biopsies were used in this study. QIAamp UltraSens™ Virus kit and EZ DNA modification kit were ordered from Qiagen (Valencia, Calif.) and Zymo Research (Orange, Calif.), respectively. Wizard DNA purification resin was obtained from Promega (Madison, Wis.). SssI CG methylase was purchased form BioLabs (Ipswich, Mass.). CpGenome Universal Methylated DNA was obtained from Chemicon International (Temecula, Calif.) and Tempase DNA polymerase was from PGC Scientific (Frederick, Md.). Deoxynucleotides were from Life Technolgies (Carlsbad, Calif.). All other chemicals were purchased from Sigma (St Louis, Mo.).

Methylation analysis: Free DNA was extracted from 1 ml of retrospectively collected serum samples using the QIAamp UltraSens™ Virus kit according to the manufacturer's protocol. The isolated DNA was subjected to chemical modification as reported by Herman et al. (25) with modification. Briefly, the DNA was denatured by 0.3 M NaOH for 15 min at 37° C. immediately before the addition of freshly prepared sodium bisulfite (3.1 M, pH 5.0) and hydroquinone (0.5 mM). The reaction was carried out at 55° C. for 16 h. The DNA was then purified using Wizard DNA purification resin, eluted to 50-μl H₂O, and incubated with NaOH (0.3 M) for 15 min at 37° C. to complete the modification. Following ethanol precipitation, the modified DNA was resuspended in 30-μl H₂O and used immediately or stored at −20° C. for one month at most. The chemically modified DNA was then analyzed in duplicates and was subjected to the “Hot Start” PCR using Tempase DNA polymerase at the presence of the M primer set in one of the duplicate and the U primer set in the other. The M primer set was specific for the methylated DNA and the U primer set was specific for the unmethylated DNA. The primer sequences of all genes for both methylated and unmethylated forms, annealing temperature and MgCl₂ concentration, cycle number, and the PCR product size were detailed in Table 1.

TABLE 1

| | | | | |
|---|---|---|---|---|
| | | SEQ | | SEQ |
| Gene | Forward Primer | ID NO: | Reverse Primer | ID NO: |

| | | | | |
|---|---|---|---|---|
| GSTP1 | M: 5′-TTCGGGGTGTAGCGGTCGTC-3′ | 1 | M: 5′-GCCCCAATACTAAATCACGACG-3′ | 2 |
| | U: 5′-GATGTTTGGGGTGTAGTGGTTGTT-3′ | 3 | U: 5′-CCACCCCAATACTAAATCACAACA-3′ | 4 |
| RASSF1A | M: 5′-GGGTTTTGCGAGAGCGCG-3′ | 5 | M: 5′-GCTAACAAACGCGAACCG-3′ | 6 |
| | U: 5′-GGTTTTGTGAGAGTGTGTTTAG-3′ | 7 | U: 5′-CACTAACAAACACAAACCAAAC-3′ | 8 |
| CDH1 | M: 5′-GGTGAATTTTTAGTTAATTAGCGG | 9 | M: 5′-CATAACTAACCGAAAACGCCGTAC-3′ | 10 |
| | U: 5′-GGTAGGTGAATTTTTAGTTAATTAGTGGTA-3′ | 11 | U: 5′-ACCCATAACTAACCAAAAACACCA-3′ | 12 |
| RARβ | M: 5′-TCGAGAACGCGAGCGATTCG-3′ | 13 | M: 5′-GACCAATCCAACCGAAACGA-3′ | 14 |
| | U: 5′-TTGAGAATGTGAGTGATTTGA-3′ | 15 | U: 5′-AACCAATCCAACCAAAACAA-3′ | 16 |
| APC | M: 5′-TATTGCGGAGTGCGGGTC-3′ | 17 | M: 5′-TCGACGAACTCCCGACGA-3′ | 18 |
| | U: 5′-GTGTTTTATTGTGGAGTGTGGGTT-3′ | 19 | U: 5′-CCAATCAACAAACTCCCAACAA-3′ | 20 |

| | | | |
|---|---|---|---|
| Gene | MgCl₂ | Temp | Cycle |

| | | | |
|---|---|---|---|
| GSTP1 M | 1.5 mM | 59° C. | 50 |
| GSTP1 U | 3.5 mM | 59° C. | 50 |
| RASSF1A M | 2.5 mM | 60° C. | 45 |
| RASSP1A U | 2.5 mM | 55° C. | 42 |
| CDH1 M | 1.5 mM | 57° C. | 55 |
| CDH1 U | 2 mM | 57° C. | 55 |
| RARβ M | 1.5 mM | 55° C. | 50 |
| RARβ U | 2.5 mM | 50° C. | 50 |
| APC M | 3.5 mM | 57° C. | 45 |
| AFC U | 1.5 mM | 50° C. | 45 |

The PCR mixture (25 μl) contained deoxynucleotides (500 nM each dNTP), primers (100 nM each), 1 unit of Tempase and 2 μl bisulfate-modified DNA. Amplification was carried out in a MJ Research PTC-200 DAN Engine (Watertown, Mass.). In each reaction, the bisulfite-modified, SssI-treated lymphocyte DNA from a healthy male donor, who had PSA level <0.5 ng/ml and had normal DRE results, was used as the methylated DNA control and its non-SssI-treated counterpart was used as the unmethylated DNA control. In some experiments, the SssI-treated lymphocyte DNA was replaced by CpGenome Universal Methylated DNA. At the end of reaction, 15 μl of the MS-PCR products were analyzed in a 2% agarose gel and visualized under UV light. The methylation specificity of the PCR was validated in each reaction by the presence of a visible “M” band in the PCR products from the reaction where the methylated DNA control was used and only a visible “U” band was presented in the PCR products from the reaction where the unmethylated DNA control was used. As to the specimens, as long as a visible “M” band was detected, either present alone (in the case of both alleles were hypermethylated) or co-present with “U” band (only one allele was hypermethylated), the gene is to be considered as hypermethylated. All samples in this study were analyzed in a double masked manner.

The DNA modification method described above had been replaced by using EZ DNA modification kit for the last 53 serum DNA samples. In both methods, bisulfit treatment was used. The modification efficiency of EZ DNA modification kit had been evaluated before used. Our comparison test confirmed that the DNA modified by two different modification methods generated comparable results as analyzed by MS-PCR.

Statistical analysis: Statistical analysis was performed using chi-square 2×2 contingency table for difference between the means of two groups. A P value <0.05 was regarded as statistically significant.

Results

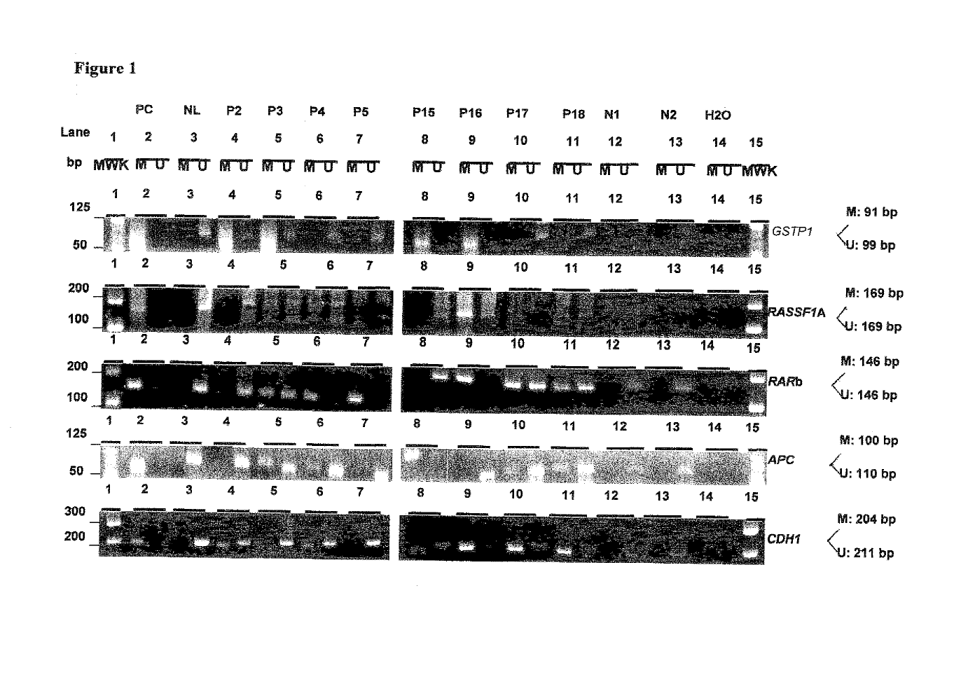

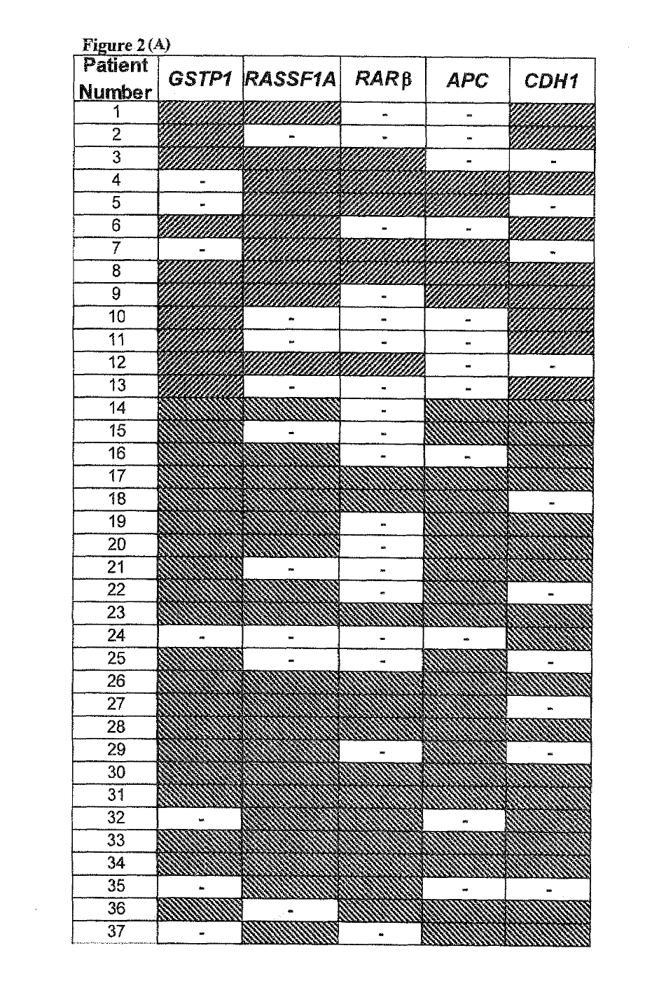

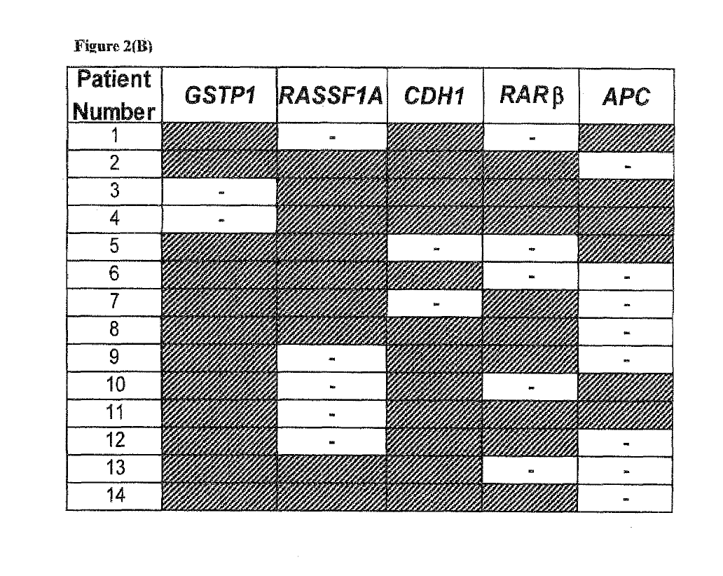

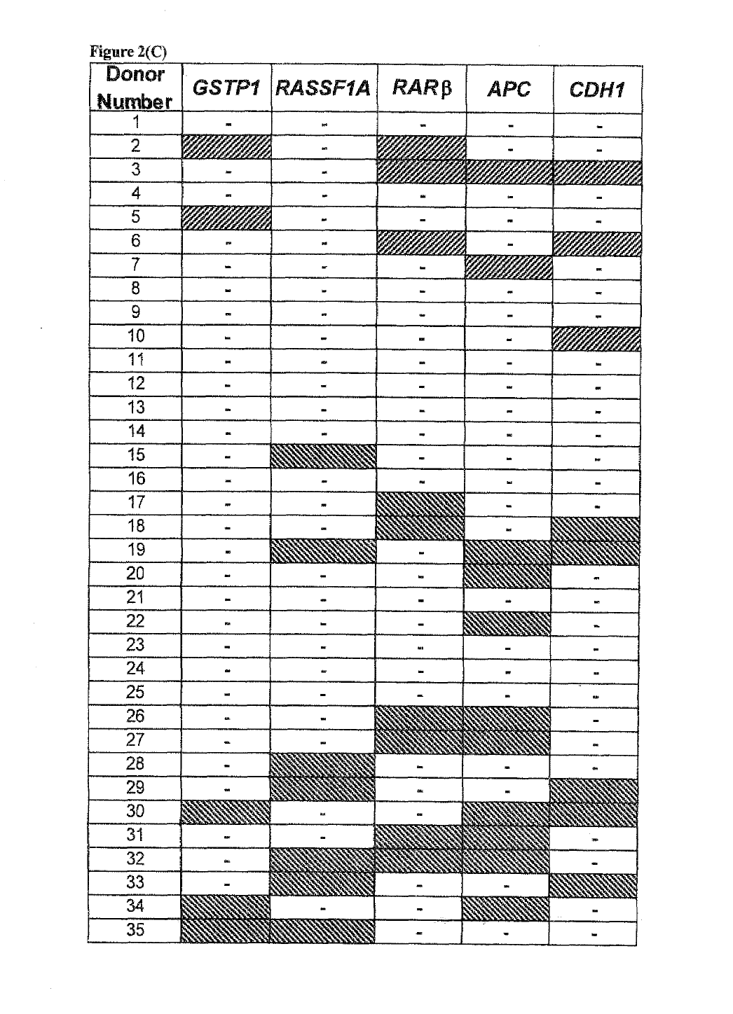

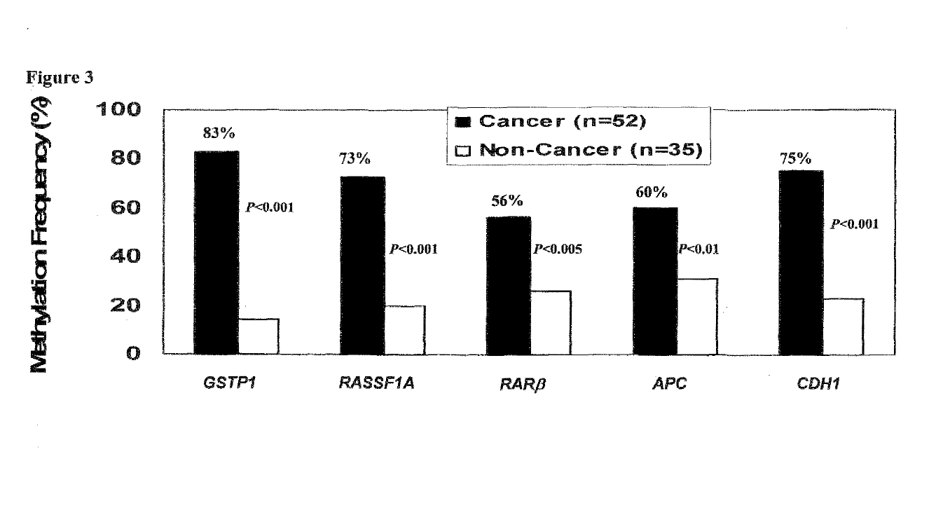

To determine the presence of cancer-specific hypermethylation; we examined serum DNA methylation of selected genes. Initially, we analyzed 87 specimens including 38 men with either clinically or pathologically organ confined prostate cancer, 14 men with proven metastatic prostate cancer and 35 men who were biopsy negative. The methylation status of the 5 selected genes, namely, GSTP1, RASSF1A, RARβ, APC and CDH1 was analyzed and the results were shown in FIGS. 1 and 2. FIG. 1 shows a representative gel analysis of MS-PCR products. The methylation status of the genes was summarized in FIG. 2. The filled boxes represents samples in which the specific genes were methylated; whereas the open boxes represent samples that are not methylated. The results clearly showed that there were significantly greater number of filled boxes in samples from the cancer patients (FIGS. 2A & B) compared to that of non-cancer group (FIG. 2C), suggesting there was a differential methylation profile between the cancer and the non-cancer groups. Our results also revealed that there was no significant difference in the methylation profile between the low- and high-grade tumors (FIGS. 2A & B), suggesting that the epigenetic abnormalities could occur at the early stage of cancer development. When the rate of methylation among the population was analyzed, the data demonstrated that all of these five genes had significantly higher (P<0.01) methylation frequency in cancer group compared to the non-cancer group (FIG. 3). Moreover, among the five genes, GSTP1, RASSF1A, and CDH1 were the 3 prevailingly methylated genes. They not only had highest methylation frequency of 83%, 73% and 75%, respectively, but also demonstrated the most significant difference statistically (P<0.001) when compared to their non-cancer counterparts. Interestingly, when we examined the methylation status of the three prevailingly methylated genes, we found that at least two of o the three genes were hypermenthylated in greater than 90% of the cancer population. Such a phenomenon is not seen in the non-cancer group (only 14.3%) in our study and not in any other types of tumors as reported by others, suggesting that such a pattern of the 3-gene panel may represent an unique methylation profile specific to prostate tumor. Thus, we hypothesize that the concurrence of DNA hypermethylation at two or more of GSTP1, RASSF1A and CDH1 genes is indicative of prostate cancer development.

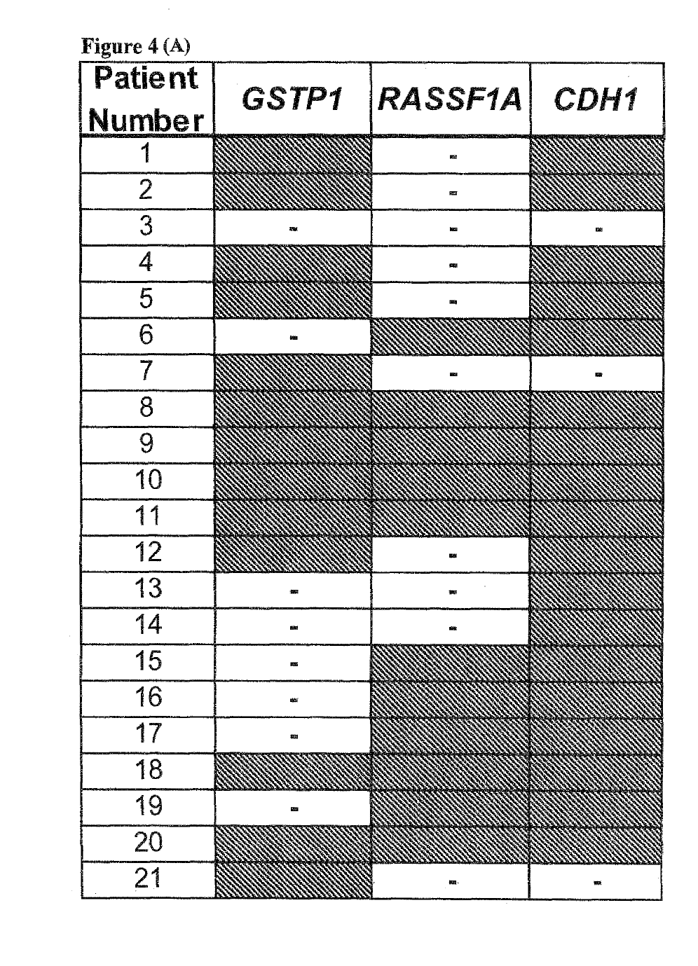

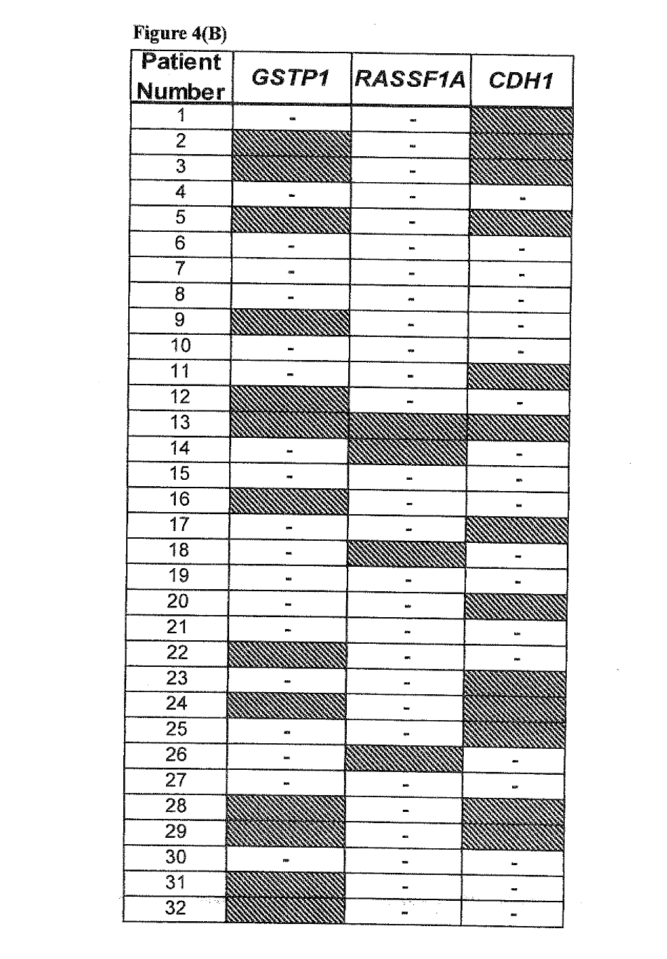

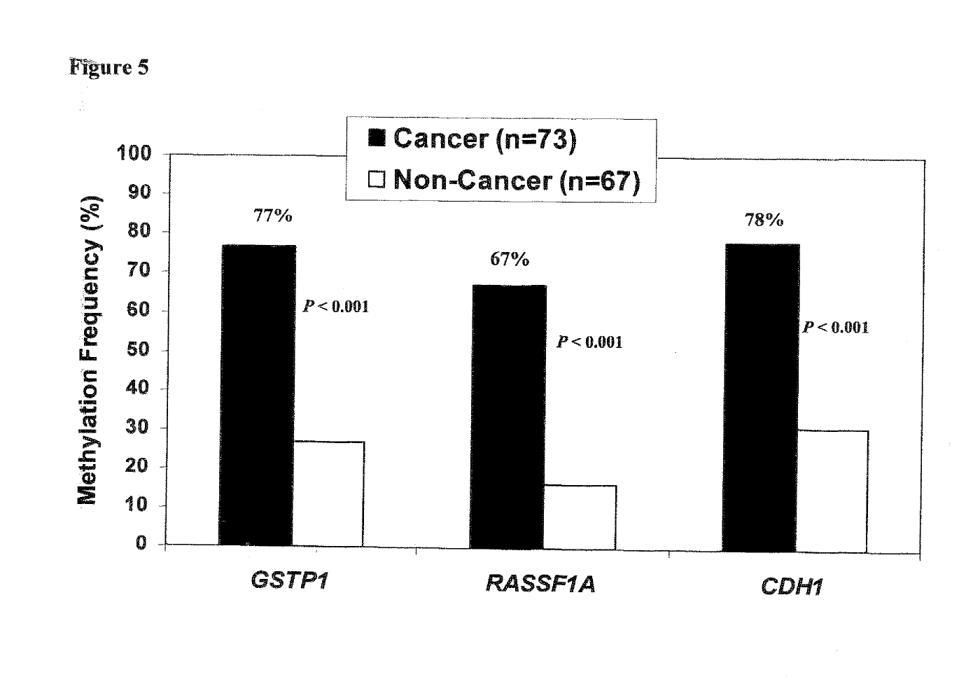

To further investigate the selective methylation of the 3-gene panel in patients with prostate cancer, we examined additional 53 individuals including 21 men who had histologically confined prostate cancer and 32 men who were extended biopsy (10 core) negative. We also narrowed down our analysis to the 3 most frequently methylated genes, i.e., GSTP1, RASSF1A and CDH1, and the results were shown in FIG. 4. Consistent with our initial findings, the methylation frequency across the 3 genes remained high among the 21 men with prostate cancer. They were 62%, 52% and 86% for GSTP1, RASSF1A, and CDH1, respectively. Together, these data and those of the initial 87 samples, the methylation frequency for the 3 genes over the 170 specimens was shown in FIG. 5. In consistent with our initial findings, the data clearly demonstrated that the methylation frequencies of the 3 prevailing genes were 67% in cancer patients and that the rates were all significantly higher (P<0.001) in the cancer group than its non-cancer counterpart.

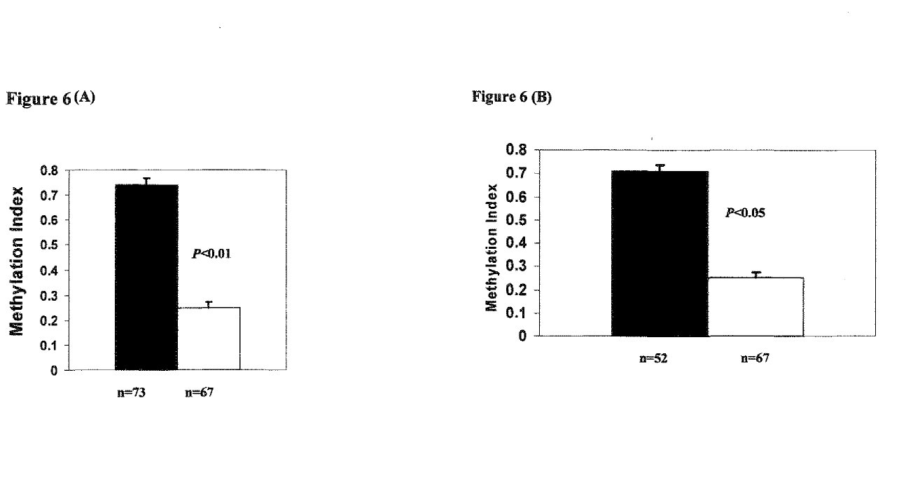

We selected a methylation index (MI) for the measurement of the extent of methylation among the genes. In this context, the MI is defined as the fraction of genes methylated to the 3 genes tested. The results show that the MI values differ significantly (P<0.01) between the means of the cancer (0.74±0.028) and non-cancer (0.25±0.023) groups (FIG. 6A). Based on our hypothesis, we further assumed a MI value of ≧0.67 as indicative of the detection of cancer. Using a MI value ≧0.67 as a cutoff value, the current data revealed an overall sensitivity (the rate of true positive) and specificity (the rate of true negative) of 86.3% and 82.1%, respectively, with a diagnostic accuracy of 91.4%. To assess the feasibility of using DNA methylation profiling for the early detection of prostate cancer, we compared the MI between the cancer and non-cancer groups whose preoperative serum PSA levels were less than 10 ng/ml. Based on such a criterion, our analysis was thus limited to the 119 men. Like the results found in the overall PSA ranges, the MI values in this low PSA subgroup were also significantly higher (P<0.05) in the cancer patients than the non-cancer group (FIG. 6B). At MI value of ≧0.67, we also observed a sensitivity of 82.7% and maintained a specificity of 82.1%.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Catalona W J, Smith D S, Ratliff T L, Dodds K M, Coplen D E, Yuan J J, Petros J A, Andriole G L. Measurement of prostate-specific antigen in serum as a screening test for prostate cancer. N Engl J Med, 324:1156-1161, 1991.
2. Weir H K, Thun M J, Hankey B F, Ries L A, Howe H L, Wingo P A, Jemal A, Ward E, Anderson R N, Edwards B K. Annual report to the nation on the status of cancer, 1975-2000, featuring the uses of surveillance data for cancer prevention and control. J Natl Cancer Inst, 95: 1276-1299, 2003.
3. Babaian R J, Camps J L, Frangos D N, Ramirez E I, Tenney D M, Hassell J S, Fritsche A, Jr: Monoclonal prostate-specific antigen in untreated prostate cancer: Relationship to clinical stage and grade. Cancer 67:2200-06, 1991.
4. Babaian R J, Miyashita H, Evans R B, Ramirez E I: The distribution of prostate specific antigen in men without clinical or pathological evidence of prostate cancer: Relationship to gland volume and age. J Urol 147:837-840, 1992.
5. Catalona W J, Partin A W, Slawin K M, Brawer M K, Flanigan R C, Patel A, Richie J P, deKernion J B, Walsh P C, Scardino P T, Lange P H, Subong E N, Parson R E, Gasior G H, Loveland K G, Southwick P C. Use of the percentage of free prostate-specific antigen to enhance differentiation of prostate cancer from benign prostatic disease: a prospective multicenter clinical trial. JAMA. 1998 May 20; 279(19):1542-7.
6. Benson M C, Whang I S, Olsson C A, McMahon D J, Cooner W H. The use of prostate specific antigen density to enhance the predictive value of intermediate levels of serum prostate specific antigen. J Urol. 1992; 147(3 Pt 2):817-21.
7. Babaian R J, Naya Y, Cheli C, Fritsche H A. The detection and potential economic value of complexed prostate specific antigen as a first line test. J Urol. 2006 March; 175(3 Pt 1):897-901; discussion 901.
8. Partin A W, Brawer M K, Bartsch G, Horninger W, Taneja S, Lepor H, Babaian R J, Childs S, Stamey T, Fritsche H A, Sokoll L, Chan D W, Thiel R P, Cheli C. Vol 170(5), November 2003, pp 1787-1791.
9. D′Amico A, Renshaw A, Sussman B, Chen M H. Pretreatment PSA velocity and risk of death from prostate cancer following external beam radiation therapy. Vol 294(4), 27 July 2005, p 440-447.
10. Carter H B, Pearson J D. PSA velocity for the diagnosis of early prostate cancer. A new concept. Urol Clin North Am. 1993 November;20(4):665-70.
11. Thomas A, Caldwell M, McNeal J S, Nolley R, Hemenez M, Downs J. The prostate specific antigen era in the United States is over for prostate cancer: What happened in the last 20 years? J Urol 172 (4) Part 1, October 2004, Pages 1297-1301.
12. Jones P A, Baylin S B. The fundamental role of epigenetic events in cancer. Nat Rev Genet 2002;3:415-428
13. Lee W H, Morton R A, Epstein J I, Brooks J D, Campbell P A, Bova G S, Hsieh W S, Isaacs W B, Nelson W G. Cytidine methylation of regulatory sequences near the pi-class glutathione S-transferase gene accompanies human prostatic carcinogenesis. Proc Natl Acad Sci USA. 1994 Nov. 22;91(24):11733-7
14. Chu D, Chuang C, Fu F, Huang H, Tseng C, Sun C. The use of real-time quantitative polymerase chain reaction to detect hypermethylation of the CpG islands in the promoter region flanking the gstpl gene to diagnose prostate carcinoma. Vol 167 (4), April 2002, Pages 1854-1858.
15. Santourlidis S, Florl A, Ackermann R, Wirtz H C, Schulz W A. High frequency of alterations in DNA methylation in adenocarcinoma of the prostate. Prostate. 1999 May 15;39(3):166-74.
16. Lin X, Tascilar M, Lee W, Vles W J, Lee B H and Veeraswamy R et al., GSTP1 CpG island hypermethylation is responsible for the absence of GSTP1 expression in human prostate cancer cells, Am J Pathol 159 (2001), p. 1815.
17. Bastian P J, Yegnasubramanian S, Palapattu G S, Rogers C G, Lin X, De Marzo A M, Nelson W G. Molecular biomarker in prostate cancer: the role of CpG island hypermethylation. Eur Urol. 2004 December;46(6):698-708.
18. Yegnasubramanian S, Kowalski J, Gonzalgo M L, Zahurak M, Piantadosi S, Walsh P C, Bova G S, De Marzo A M, Isaacs W B, Nelson W G. Hypermethylation of CpG islands in primary and metastatic human prostate cancer. Cancer Res. 2004 Mar 15;64(6):1975-86.
19. Jeronimo C, Henrique R, Campos P F, Oliveira J, Caballero O L, Lopes C, Sidransky D. Endothelin B receptor gene hypermethylation in prostate adenocarcinoma. J Clin Pathol. 2003 January;56(1):52-5.
20. Jeronimo C, Usadel H, Henrique R, Silva C, Oliveira J, Lopes C, Sidransky D. Quantitative GSTP1 hypermethylation in bodily fluids of patients with prostate cancer. Urology. 2002 December;60(6):1131-5.
21. Goessl C, Krause H, Muller M, Heicappell R, Schrader M, Sachsinger J, Miller K. Fluorescent methylation-specific polymerase chain reaction for DNA-based detection of prostate cancer in bodily fluids. Cancer Res. 2000 Nov. 1;60(21):5941-5.
22. Goessl C, Muller M, Heicappell R, Krause H, Miller K. DNA-based detection of prostate cancer in blood, urine, and ejaculates. Ann N Y Acad Sci. 2001 September;945:51-8
23. Papadopoulou E, Davilas E, Sotiriou V, Koliopanos A, Aggelakis F, Dardoufas K, Agnanti N J, Karydas I, Nasioulas G. Cell-free DNA and RNA in plasma as a new molecular marker for prostate cancer. Oncol Res. 2004;14(9):439-45.
24.Vis A N, Oomen M, Schroder F H, van der Kwast T H. Feasibility of assessment of promoter methylation of the CD44 gene in serum of prostate cancer patients. Mol Urol. 2001 Winter; 5(4):199-203.
25. Herman J G, Graff J R, Myohanen S, Nelkin B D, and Baylin S B. Methylation-specific PCR: a novel PCR assay for methylation status of CpG islands. Proc Natl Acad Sci USA, 93: 9821-9826, 1996.

What is claimed is:

1. A method of screening a mammal for a cancer associated with methylation of promoters of tumor-suppressor genes, comprising:
collecting a sample of a body fluid or tissue of the mammal containing free DNA;
isolating the free DNA from the sample;
amplifying at least a portion of the promoter of each of a plurality of tumor-suppressor genes associated with the cancer from the free DNA, to yield a plurality of amplified promoters; and
quantifying methylation of each of the plurality of amplified promoters, to yield a methylation quantity.

2. The method of claim 1, further comprising comparing the methylation quantity to a threshold quantity.

3. The method of claim 1, wherein the mammal is Homo sapiens.

4. The method of claim 3, wherein the cancer is prostate cancer.

5. The method of claim 4, wherein the plurality of tumor-suppressor genes associated with the cancer are selected from the group consisting of GSTP1, RASSF1A, and CDH1.

6. The method of claim 1, wherein the body fluid or tissue is selected from the group consisting of blood and urine.

7. The method of claim 1, wherein amplifying comprises methylation-specific polymerase chain reaction (MS-PCR).

8. The method of claim 7, wherein MS-PCR comprises the use of two or more sets of primers, each set selected from the group consisting of SEQ ID NO:1-4, SEQ ID NO:5-8, SEQ ID NO:9-12, SEQ ID NO:13-16, and SEQ ID NO:17-20.

9. The method of claim 7, wherein quantifying comprises dividing the number of methylated amplified promoters by the total number of promoters subjected to amplification.

10. The method of claim 1, wherein amplifying comprises methylation-specific polymerase chain reaction (MS-PCR) and pyrosequencing.

11. The method of claim 10, wherein MS-PCR comprises the use of two or more sets of primers, each set selected from the group consisting of SEQ ID NO:1-4, SEQ ID NO:5-8, SEQ ID NO:9-12, SEQ ID NO:13-16, and SEQ ID NO:17-20.

12. The method of claim 10, wherein quantifying comprises dividing the cytosine peak height by the (cytosine+tyrosine) peak height.

13. A system for screening a mammal for a cancer associated with methylation of promoters of tumor-suppressor genes, comprising:
means for collecting a sample of a body fluid or tissue of the mammal containing free DNA;
means for isolating the free DNA from the sample;
means for amplifying at least a portion of the promoter of each of a plurality of tumor-suppressor genes associated with the cancer from the free DNA; and
means for quantifying methylation of each of the plurality of amplified promoters.


SEQUENCE LISTING (20 sequences)

SEQ ID NO: 1 - DNA, 20 bp, artificial sequence
  Forward primer for PCR amplification of methylated human GSTP1 promoter
ttcggggtgtagcggtcgtc

SEQ ID NO: 2 - DNA, 22 bp, artificial sequence
  Reverse primer for PCR amplification of methylated human GSTP1 promoter
gccccaatactaaatcacgacg

SEQ ID NO: 3 - DNA, 24 bp, artificial sequence
  Forward primer for PCR amplification of unmethylated human GSTP1 promoter
gatgtttggggtgtagtggttgtt

SEQ ID NO: 4 - DNA, 24 bp, artificial sequence
  Reverse primer for PCR amplification of unmethylated human GSTP1 promoter
ccaccccaatactaaatcacaaca

SEQ ID NO: 5 - DNA, 18 bp, artificial sequence
  Forward primer for PCR amplification of methylated human RASSF1A promoter
gggttttgcgagagcgcg

SEQ ID NO: 6 - DNA, 18 bp, artificial sequence
  Reverse primer for PCR amplification of methylated human RASSF1A promoter
gctaacaaacgcgaaccg

SEQ ID NO: 7 - DNA, 22 bp, artificial sequence
  Forward primer for PCR amplification of unmethylated human RASSF1A promoter
ggttttgtgagagtgtgtttag

SEQ ID NO: 8 - DNA, 22 bp, artificial sequence
  Reverse primer for PCR amplification of unmethylated human RASSF1A promoter
cactaacaaacacaaaccaaac

SEQ ID NO: 9 - DNA, 27 bp, artificial sequence
  Forward primer for PCR amplification of methylated human CDH1 promoter
ggtgaatttttagttaattagcggtac

SEQ ID NO: 10 - DNA, 21 bp, artificial sequence
  Reverse primer for PCR amplification of methylated human CDH1 promoter
cataactaaccgaaaacgccg

SEQ ID NO: 11 - DNA, 30 bp, artificial sequence
  Forward primer for PCR amplification of unmethylated human CDH1 promoter
ggtaggtgaatttttagttaattagtggta

SEQ ID NO: 12 - DNA, 24 bp, artificial sequence
  Reverse primer for PCR amplification of unmethylated human CDH1 promoter
acccataactaaccaaaaacacca

SEQ ID NO: 13 - DNA, 20 bp, artificial sequence
  Forward primer for PCR amplification of methylated human RARbeta promoter
tcgagaacgcgagcgattcg

SEQ ID NO: 14 - DNA, 20 bp, artificial sequence
  Reverse primer for PCR amplification of methylated human RARbeta promoter
gaccaatccaaccgaaacga

SEQ ID NO: 15 - DNA, 21 bp, artificial sequence
  Forward primer for PCR amplification of unmethylated human RARbeta promoter
ttgagaatgtgagtgatttga

SEQ ID NO: 16 - DNA, 20 bp, artificial sequence
  Reverse primer for PCR amplification of unmethylated human RARbeta promoter
aaccaatccaaccaaaacaa

SEQ ID NO: 17 - DNA, 18 bp, artificial sequence
  Forward primer for PCR amplification of methylated human APC promoter
tattgcggagtgcgggtc

SEQ ID NO: 18 - DNA, 18 bp, artificial sequence
  Reverse primer for PCR amplification of methylated human APC promoter
tcgacgaactcccgacga

SEQ ID NO: 19 - DNA, 24 bp, artificial sequence
  Forward primer for PCR amplification of unmethylated human APC promoter
gtgttttattgtggagtgtgggtt

SEQ ID NO: 20 - DNA, 22 bp, artificial sequence
  Reverse primer for PCR amplification of unmethylated human APC promoter
ccaatcaacaaactcccaacaa